(19) United States
(12) Patent Application Publication
(10) Pub. No.: US 2012/0244103 A1
(43) Pub. Date: Sep. 27, 2012

(54) REVERSIBLE ORAL ADHESIVE GEL

(76) Inventors: Robert J. Davis, Palm Beach (AU); John Alexander Staton, Kingsgrove (AU); George John Orban, Eastwood (AU)

(21) Appl. No.: 13/394,109

(22) PCT Filed: Aug. 30, 2010
(86) PCT No.: PCT/IB2010/002321

§ 371 Date: Jun. 14, 2012

(30) Foreign Application Priority Data
Sep. 4, 2009 (AU) 2009904239

(51) Int. Cl. A61K 31/765; A61K 31/79; A45D 40/26; C09J 139/06; B65D 85/00; A61P 11/00; C09J 135/08

(52) U.S. Cl. 424/78.02; 526/271; 526/264; 206/349; 401/292

(57) ABSTRACT

The invention relates to a reversible oral adhesive gel. The reversible oral adhesive gel is suitable for application to lips to inhibit oral (mouth) breathing and to promote nasal breathing and thereby prevent or ameliorate snoring and to correct other respiratory problems.

### CROSS REFERENCE TO RELATED APPLICATIONS

This application claims priority to Australian Provisional Patent Application No. 2009904239 filed on Sep. 4, 2009. The application is incorporated herein by reference in its entirety.

### TECHNICAL FIELD

The present invention relates to a reversible oral adhesive gel. The reversible oral adhesive gel is suitable for application to lips to inhibit oral (mouth) breathing and to promote nasal breathing and thereby prevent or ameliorate snoring and to correct other respiratory problems.

### BACKGROUND OF THE INVENTION

Snoring is a widespread problem with significant social and medical consequences. It is estimated that more than 45% of adult men and 30% of adult women suffer from snoring. Some snorers have no symptoms and only become aware that they snore because of the feedback they receive from their sleep-deprived bed partner. Although the individual who snores may be unaware of the problem, their sleeping partner will be all too aware of the frustration and sleep deprivation that can result from their partners snoring. Besides being unfair to the non-snoring partner, if this problem is not dealt with, snoring can lead to a strained relationship and loss of intimacy. However, for many snorers, the direct consequences to their health and well-being can be quite significant. Symptoms resulting from snoring can include being repeatedly awoken from sleep, morning headaches, chronic fatigue, irritability, poor work performance, decreased libido, weight gain and depression as well as having increased risk of developing sleep apnea, diabetes, hypertension and cardiovascular disease.

Snoring is the harsh sounds that result from the vibration of soft tissues (primarily the soft palate and uvula) due to turbulent airflow in the throat and upper airway. During sleeping, the soft tissues in the throat and upper airway relax leading to constricted airflow and vibration of the surrounding soft tissues. The loudness and frequency of snoring occurs across a spectrum from mild and intermittent to chronic and severe. As many as 50% of severe snorers may have or eventually develop a serious health condition called obstructive sleep apnea. Any factors that lead to narrowing of the airway and/or increase airflow turbulence can predispose to snoring. These can include:
Sleeping on your back (tongue falls backward→narrowed airway)
Alcohol (muscle relaxant→narrowed airway)
Sedatives, anti-depressants (muscle relaxants→narrowed airway)
Obesity (especially fat deposits around neck→narrowed airway)
Aging (increased laxity of soft tissues→narrowed airway)
Nasal obstruction (infection, polyps, deviated septum→increased mouth breathing)
Mouth breathing
   Increased airflow turbulence
   Tongue falls backward→narrowed airway

Mouth breathing is a major contributing factor in many individuals who suffer from snoring. Mouth breathing causes an increase in airflow turbulence (compared to nasal breathing) and narrowing of the airway due to posterior migration of the base of the tongue. Both of these factors contribute to and exacerbate the severity of snoring. Mouth breathing also predisposes to dryness of the lips and mouth, halitosis, mouth ulcers, post nasal drip, dental conditions and facial deformities.

In addition to being a significant cause of snoring, mouth breathing also undermines the important health benefits provided by nasal breathing. Nasal breathing enables air to flow into the nasal canal and allows the paranasal sinuses to filter, moisturize, warm and dehumidify inhaled air prior to its entry into the lungs. These important functions of the sinuses help to fight infection and ensure that the lungs receive an infusion of high quality air.

A number of problems can compromise nasal breathing including upper respiratory infections (ie, colds, sinusitis), allergies, nasal polyps and a deviated nasal septum. Chronic impairment of nasal breathing can significantly contribute to snoring as well as predisposing to chronic sinusitis and having potentially adverse consequences for pulmonary function including the development of and/or exacerbation of asthma. Aside from increasing the likelihood of snoring, breathing through the mouth, especially during sleep, prevents nasal breathing, thereby shutting off air circulation in the nasal and sinus cavities. This compromises the important physiologic functions normally provided by the paranasal sinuses. Thus, prevention of mouth breathing helps to restore the important health benefits normally provided by nasal breathing.

A myriad of approaches have been used to treat snoring. These include eliminating sources of nasal obstruction, the use of nasal rinses, natural remedies such as herbs, acupressure, or acupuncture, the use of antidepressants or other drugs, losing weight, stopping smoking, limiting alcohol and sedative use prior to sleeping, avoiding sleeping on the back, the application of splints or strips to the soft palate, teeth or nose, the use of dental devices to seal the lips, maintain closure of the jaws and prevent posterior migration of the tongue, surgical alteration of the soft tissues in the nasal canal, throat and upper airway and the use of a continuous positive airway pressure machine. As it has become increasingly apparent that oral breathing is a major causative factor for most snorers, a major focus for snoring therapy has become the elimination of mouth breathing. This is a critical component of many of the devices that are used to support the jaw, prevent backward migration of the tongue and seal the lips.

In summary, snoring is highly prevalent and can have serious social and health consequences for the snorer and their sleeping partner. Oral breathing is a major causative factor of snoring. Oral breathing also prevents nasal breathing which can compromise the important physiologic functions provided by the paranasal sinuses. Thus, preventing oral breathing can effectively treat snoring and mitigate against its associated adverse health consequences whilst simultaneously facilitating the positive health benefits provided by nasal breathing. Prevention of mouth breathing with an easily reversible oral adhesive gel represents a safe, economical, user friendly approach for treating snoring compared to many of the other more invasive, expensive and unpleasant therapies such as drugs, devices and surgery currently used to treat snoring and mitigate against its adverse health consequences.

Australian Patent PCT/AU2007/001516 (WO 2008/043132) relates to an adhesive strip for applying to the lips, which strip is not reversibly adhesive.

### OBJECT OF THE INVENTION

It is the object of the present invention to prevent or substantially limit oral breathing, in a reversible manner, in order to treat snoring and other health care disorders that may be ameliorated by diminishing oral breathing and/or enhancing nasal breathing.

### SUMMARY OF THE INVENTION

According to a first aspect of the invention there is provided a reversible oral adhesive gel, the oral adhesive comprising at least one adhesive agent, the reversible oral adhesive in the form of a gel. Preferably, the reversible oral adhesive gel has a pH of 5.0 or lower. A reversible oral adhesive gel does not include an adhesive strip.

According to one aspect of the invention, the reversible oral adhesive gel has an adhesive strength strong enough to hold two surfaces together but not strong enough to resist removal without causing damage. For example, the oral adhesive is suitably strong enough to hold a person's lips together while sleeping but not too strong so as to cause damage if the user opens their mouth without first pushing the tongue through the lips. Suitably the reversible oral adhesive gel has an average maximum stress value ranging from about 0.03 to about 0.1 MPa. A liquid or semi-liquid adhesive may be used.

In one embodiment more than one reversible adhesive agent is used, e.g., from the adhesive agents methyl vinyl ether/maleic anhydride copolymer, or mixed sodium/calcium salts thereof and soluble polyvinyl pyrrolidone In one embodiment the reversible adhesive agent is mixed sodium/calcium salts of methyl vinyl ether/maleic anhydride copolymer, such as sold under the trade name Gantrez®, including the adhesive Gantrez® MS-955 and soluble polyvinyl pryyolidones, such as sold under the trade name Kollidon®, including the adhesive Kollidon® 90F. Oral adhesives and methods for producing them are described, for example, in U.S. Pat. Nos. 5,369,145; 5,525,652; 5,561,177; 5,750,591; 6,025,411; 6,110,989; 6,239,191; 6,423,762, which are herein incorporated by reference.

The reversible oral adhesive gel may further comprise one or more of: one or more chelating agents such as ethylene diamine tetraacetic acid or sodium or potassium salts thereof. In one embodiment the chelating agent is disodium edetate; one or more preservatives such as alkyl hydroxybenzoate or their salts, sorbic acid or its salts, benzoic acid or its salts or other suitable ingestible preservatives familiar to those skilled in the art. One suitable preservative is potassium sorbate; one or more pH adjusters such as anhydrous citric acid. It will be appreciated by those skilled in the art that, depending on the final choice of preservative, an appropriate pH adjustment may need to be made. It will also be appreciated that in some cases, a pH adjustment may not be necessary; one or more hydrophilic gelling/thickening agents such as xanthan gum, carageenan gum, guar gum, sodium alginate, acacia gum, hydroxyethylcellulose, hydroxypropylcellulose, hydroxypropylmethylcellulose, carboxymethylcellulose or sodium salts thereof, gelatin or pectin. In one embodiment the hydrophilic gelling agent/thickener is xanthan gum. In one embodiment, the reversible oral adhesive gel comprises xanthan gum as a hydrophilic gelling agent and mixed sodium/calcium salts of methyl vinyl ether/maleic anhydride copolymer as the adhesive agent; one or more additional thickeners such as silicon dioxide (for example Aerosil® 200), polyacrylamide/C₁₃-C₁₄ isoparrafin/laureth-7 (for example Sepigel® 305), acrylamide copolymer/mineral oil/C₁₃-C₁₄ isoparrafin/polysorbate 85; one or more lubricants such as a dimethicone. In one embodiment the lubricant has a viscosity of 200 cps such as is available as Dow Corning200® Fluid 200 cs.

The lubricants also act as water resistance imparting agents; one or more solvents such as ethanol 95% and/or one or more diluents such as an aqueous base or water.

The adhesive agent or agents may be present in an amount of from about 5 to about 25 wt %, for example from about 10 to about 20 wt %, for example about 5 wt %, about 10 wt %, about 15 wt %, about 20 wt % or about 25 wt %.

The chelating agent may be present in an amount of about 0.01 to 0.2 wt %, for example about 0.1 wt %.

The preservative may be present in an amount of about 0.01 to 1 wt %, for example about 0.2 wt %.

The pH adjuster, when present, is added in a sufficient quantity to obtain a final pH of the composition of 5.0 or lower. For example the pH adjuster may be present in amount of about 2 wt %.

The thickener/hydrophilic gelling agent may be present in an amount of about 1 wt % to 10 wt %, for example 1 wt %, 1.5 wt % or 4 wt %.

In one embodiment each additional thickener may be present in an amount of 1 to 10 wt %. For example a first additional thickener, preferably Sepigel®305 may be present in an amount of 1 to 10 wt % and a second additional thickener, preferably fumed silicon dioxide such as Aerosil® 200 may be present in an amount of 1 to 10 wt %.

The lubricant may be present in an amount of from about 1 to about 10 wt %, for example about 4.5 wt %.

The diluent may be present in an amount to make up 100 wt % for example about 66.7 wt %.

A reversible oral adhesive agent according to the invention is safe for oral use in humans.

According to a second aspect of the invention there is provided a method of treating a patient or subject, the method comprising applying the reversible oral adhesive gel of the invention to the lips to aid in securing the lips of the patient or subject together to inhibit the patient or subject from breathing through their mouth and to encourage the patient or subject to breathe through their nose. In some instances this may be achieved by applying the reversible oral adhesive to the central portion of the lower lip alone, in other instances it may require application from end-to-end on the lower lip alone and in other instances it may require application to the upper lip in combination with application to the lower lip.

Typically the reversible oral adhesive gel is applied to the central part of the lower lip. The reversible oral adhesive gel is applied to the moist part of the lip, just behind the dry/moist line. Because of the nature of the sphincter muscle which controls the lips, it is sufficient to control only the central part of the lips with the gel to influence the sphincter muscle to keep the rest of the mouth-opening closed during sleep. When awake, the cheek muscle can be used to override the control of the sphincter muscle to enable oral breathing through the sides of the mouth if desired. This embodiment of the present invention that limits or prevents oral breathing by securing the central portions of the lips is novel relative to existing devices for preventing oral breathing in that said predicate devices attempt to prevent mouth breathing by preventing opening of the entire jaw and/or lips.

In other instances, if broader lip adhesion is desired, it may require application from end-to-end on the lower lip alone and in other instances may require application to the upper lip in combination with application to the lower lip.

Typically the gel is applied to the lips prior to going to sleep, suitably at nighttime. Suitably the oral adhesive is applied for about 6 to 9 hours.

In one embodiment, the gel suitably has a bond strength which secures the lips together when the mouth is closed but also allows speaking, coughing, yawning, sneezing, drinking and taking tablets by releasing and resealing the gel-induced adhesion at will for a limited number of times. For example, the reversible oral adhesive gel enables the user to detach the seal with the tongue and allows opening of the mouth to speak, cough, drink, yawn, sneeze, take tablets or open the mouth for any other reason but also may be resealed by wetting with the tongue and closing the lips. Reapplication of the adhesive may therefore not be required. The potential for reversible oral adhesion is a novel property of the invention

The gel is suitably straw-coloured so that it is barely visible with use. As the gel is applied behind the dry/moist line of the lips, it is mostly out of sight when the lips are closed.

It has been found that application of the reversible oral adhesive gel of the present invention promotes nasal breathing and prevents or reduces snoring, sinusitis, dry mouth, sleep apnea, nasal congestion, post nasal drip, bad breath, mouth ulcers, tooth decay, and reduces the severity of asthma. Without being bound to any particular theory, it is thought that breathing through the nose during sleep keeps the upper airways ventilated, reduces sinus pressure and prevents excessive drying of the oral cavity lining and saliva. The reversible oral adhesive gel of the present invention may be used to treat or avoid halitosis, tooth decay, nasal congestion, post nasal drip, bad breath, mouth ulcers and breathing problems leading to facial deformity, nasal congestion, sleep deprivation, and asthma.

The reversible oral adhesive gel may be removed from the lips as desired by application of a suitable solvent such as water or saliva. In one embodiment the oral adhesive is removed by saliva for example by pushing the tongue through the lips before opening the mouth, the saliva immediately breaking the seal. Application of sufficient additional saliva and/or water will remove the reversible oral adhesive gel. If additional saliva and/or water sufficient to remove the gel is not applied, the lips can be resealed for up to six hours from initial application by re-wetting the gel with the tongue and closing the lips.

A reversible adhesive oral gel according to the invention is packaged for use in any convenient manner for application to the lips. For example the gel may be contained in a tube comprised of aluminum or aluminum barrier laminate (ABL). Both aluminum and ABL tubes are collapsible, without memory; therefore they remain collapsed and do not return to the pre-squeezed shape which action would draw air back into the tube causing possible corruption to the adhesive quality of the gel or premature drying. Both aluminum and ABL tubes are non-porous thereby preventing corruption to the gel through osmosis. Specifications of a preferred tube are in the range of: Length: 80 mm to 120 mm; Diameter: 15 mm to 30 mm; Nozzle: extended by 6 mm to 12 mm for more accurate application to the lips; Orifice: size 1.8 mm to 2.5 mm; Cap: plastic screw type.

### BRIEF DESCRIPTION OF THE DRAWINGS

A preferred embodiment of the present invention incorporating a single adhesive agent will now be described, by way of an example only, with reference to the accompanying drawings and attachments wherein:

FIG. 1 is a stress vs. extension plot of the reversible oral adhesive gel in accordance with the invention;

FIG. 2 is a graph of the maximum bond strength of the reversible oral adhesive gel of the invention;

The safety profile of the invention are demonstrated by sensitivity and cytotoxicity studies described herein.

### DEFINITIONS

The definitions contained herein may be helpful in understanding the description of the present invention.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

As used herein, the term “reversible oral adhesive gel” means that the adhesive has sufficient strength to hold the two surfaces of the lips together for at least thirty minutes and as long as twelve hours, but is not strong enough to cause damage to the surface of the lip or lips. For example, the reversible oral adhesive gel is suitably strong enough to hold a person's lips together while sleeping but is not so strongly adhesive that it causes damage if the user opens their mouth without first pushing the tongue through the lips. Additionally, the adhesive gel is reversible in that the adhesive bond sealing the lips together can be broken by inserting the tongue between the lips and then the lips can be subsequently resealed without applying any additional adhesive gel by simply moistening the applied gel and placing the two surfaces of the lips together. For example, a person using the reversible oral adhesive gel who awakens from sleep and wishes to drink, take a pill or talk can break the seal with their tongue, complete the desired activity and then press their lips back together and the adhesive bond will be suitably restored. A reversible oral adhesive gel does not include an adhesive strip.

A simple test for determining whether an adhesive gel is “reversible” according to the invention is the following: 0.5 grams of the reversible oral adhesive gel is applied to the lips, the lips are contacted for 10 to 60 minutes to enable adherence, the tongue is inserted between the lips for at least 2 seconds to reverse the adhesion so that the lips can fully part, the gel is then moistened by water or saliva within 5 minutes, the lips are again contacted to restore the seal and may remain sealed for at least 1 hour.

“Damage”, as used here, refers to removal of a sufficient layer of the surface of the epithelium to cause chafing, soreness, and/or irritation to the average adult after a single use of the adhesive gel on the lips.

The information provided herein and references cited are provided solely to assist the understanding of the reader, and do not constitute an admission that any of the references or information is prior art to the present invention.

### DETAILED DESCRIPTION OF THE PREFERRED EMBODIMENTS

### Example 1

### First Preferred Embodiment (Single Adhesive)

A reversible oral adhesive gel incorporating a single adhesive agent was prepared according to Table 1:

TABLE 1

| w/w % | Gm | Material | Function | Supplier |
|---|---|---|---|---|
| 66.7 ie q.s to | 667 | Purified water | Diluent | |
| 100 w/w % | | | | |
| 0.1 | 1 | Disodium | chelating agent | IMCD |
| | | Edetate | | |
| 0.2 | 2 | Potassium | Preservative | IMCD |
| | | Sorbate | | |
| 2.0 ie q.s. to | 20 | Citric Acid | pH adjuster | APS |
| pH <5.0 | | Anhydrous | | Chemicals |
| 20.0 | 200 | Gantrez ® | Adhesive | ISP |
| | | MS-955 | | |
| 4.0 | 40 | Xanthan Gum | thickener/gelling | Bronson & |
| | | | agent | Jacobs |
| 4.5 | 45 | Dimethicone | Lubricant | Ingredients |
| | | 200 | | Plus |
| 1.0 | 10 | Aerosil ® 200 | Thickener | Degussa |
| 1.5 | 15 | Sepigel ® 305 | Thickener | Bronson & |
| | | | | Jacobs |

With reference to the above table, Gantrez ® MS-955 is a calcium/sodium PVM/MA copolymer, Dimethicone 200 is DC silicone fluid 200/200, Aerosil ® 200 is silicon dioxide and Sepigel ® 305 is the combination of polyacrylamide, C₁₃-C₁₄ isoparaffin and laureth-7.

Disodium edetate, potassium sorbate and citric acid were dissolved in the purified water. The Gantrez® MS-955 was added and mixed until totally dispersed avoiding any lump formation. At this stage the batch began to thicken. Xanthan gum was then added and mixed until uniform avoiding any lump formation. It was noted that the batch was further thickened by this addition. Dimethicone was then added until uniform, followed by the addition of Aerosil® 200 (with further thickening of the batch) and finally by the addition of Sepigel® 305 which was mixed until uniform (again with further thickening of the batch).

The resulting product was a thick, pale straw coloured, slightly translucent gel with a slightly gum like, otherwise bland odour. The pH of the gel was 4.0 to 5.0 (1 in 5 dilution with water) and the viscosity was about 1 million cps (as determined using a Brookfield RVT Helipath Spindle F, 5 rpm). The gel was microbiologically tested (ams TM103, TM101 and TM102) and no Pseudomonas or Staph. Aureus species were noted. The total plate count was less than 100 cfu/g, yeasts and moulds representing less than 100 cfu/g.

### Example 2

### Tensile Studies of the First Preferred Embodiment

14 samples of the oral adhesive in accordance with Example 1 were subjected to the following adhesion tests to determine the bond strength of the adhesive. Test specimens were cut from an extruded length of T-profile aluminium. Test surfaces measure approximately 40 mm×40 mm and precise dimensions were determined for each specimen. The surfaces of the test specimens were ultrasonically cleaned with soapy water and acetone prior to application of the adhesive in accordance with Example 1. The tested adhesive was applied as a uniform coating on each of two surfaces of the test specimens and the two test pieces held together to firmly bond them together. The adhesive was given 20 hours to set.

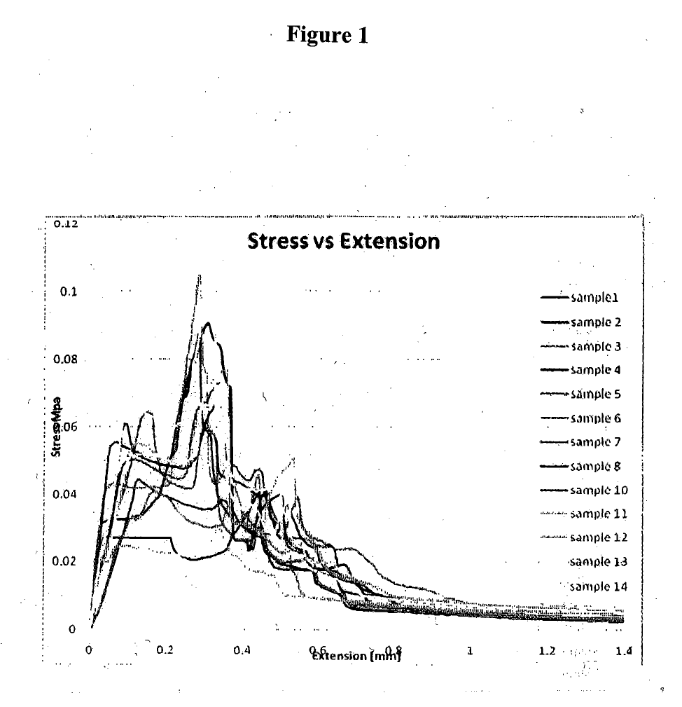

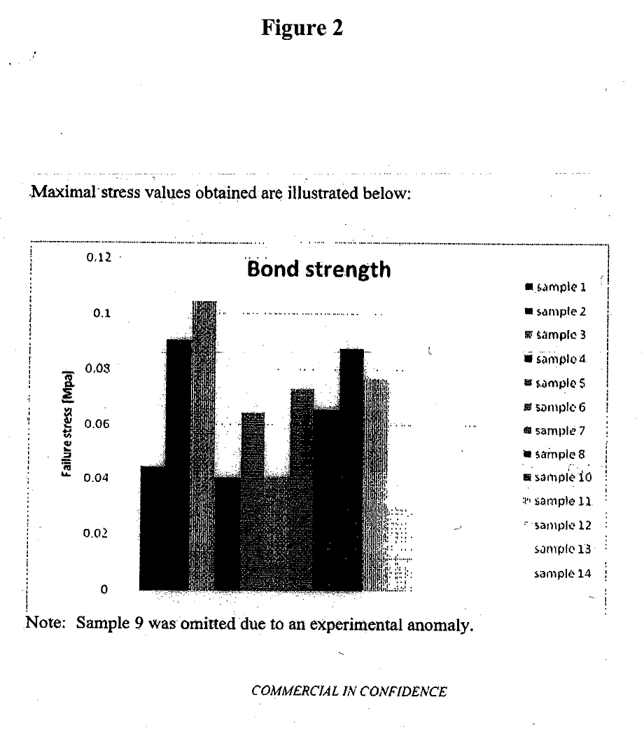

Tensile tests were carried out using an Instron 1185 mechanical testing rig (Instron, Norwood, Mass.) using a constant crosshead speed of 1 mm per minute. Output was generated as load vs crosshead extension and subsequently corrected for actual specimen dimension providing the stress vs extension plots shown in FIG. 1. Maximum stress values obtained are shown in FIG. 2. In FIG. 2, sample 9 was excluded due to an experimental anomaly. Table 2 shows the maximal stress determined for each sample:

TABLE 2

| Specimen | Maximal Stress [MPa] |
|---|---|
| 1 | 0.044565609 |
| 2 | 0.090877109 |
| 3 | 0.104878125 |
| 4 | 0.040597583 |
| 5 | 0.064454105 |
| 6 | 0.040885411 |
| 7 | 0.073077198 |
| 8 | 0.0661876 |
| 9 | 0.018191667 |
| 10 | 0.087683092 |
| 11 | 0.076724488 |
| 12 | 0.030151302 |
| 13 | 0.081838625 |

The average stress was determined to be 0.0704 MPa which is well within the desirable range of stress values.

After 20 hours curing time the adhesive was still somewhat viscous and had not hardened probably due to lack of exposure to air during the curing process. The adhesive failed gradually in a visco-elastic manner as illustrated by the jagged stress-extension curves shown in FIG. 1.

From the above it is clear that the oral adhesive showed an average bond strength of 0.0704 MPa after 20 hours of curing and while still viscous.

### Example 3

### Sensitivity Studies of the First Preferred Embodiment

Skin patch sensitivity studies were performed for 58 human subjects by Cantor Research Laboratories, Inc of Blauvelt, N.Y., USA utilizing the principles referenced in Appraisal of the Safety of Chemicals in Food, Drugs and Cosmetics published by The Association of Food and Drug Officials of the United States.

The following procedure was used to evaluate irritation/sensitization: Subjects included in the study were individuals free of any dermatological or systemic disorder which would have interfered with the results. Individuals who were currently taking any medication (topical or systemic) that may have masked or interfered with the test results were excluded. Demographics of the subjects are show in Table 3.

TABLE 3
Sensitivity Study Population Demographics

| | |
|---|---|
| Number of subjects enrolled | 58 |
| Number of subjects completing study | 57 |
| Age Range | 20-66 |
| Sex | |
| Male | 13 |
| Female | 45 |
| Race | |
| Caucasian | 24 |
| Hispanic | 4 |
| Asian | 2 |
| African American | 28 |

A patch containing the test material was applied directly to the skin of the infrascapular regions of the back, to the right or left of the midline of a subject. 0.2 ml of the test material was dispensed onto a semi-occlusive, hypoallergenic patch (Parke-Davis Hypoallergenic Readi Bandages (20×20 mm Webril affixed to the center of a 40×40 mm adhesive bandage) or the equivalent, trimmed at right angles on opposite sides to the opening of the paper backing of patch, allowing air flow). The subject was dismissed with instructions not to wet or expose the test area to direct sunlight. After 24 hours, the patch was removed by the panelist at home. This procedure was repeated three days per week (every Monday, Wednesday and Friday) for three consecutive weeks until a series of nine consecutive 24 hour applications were made.

In the event of an adverse reaction, the area of erythema and edema was measured. The edema was estimated by the evaluation of the skin with respect to the contour of the unaffected normal skin. Reactions were scored just before applications two through nine and the next test date following application nine. In the case of adverse reaction, determination was made as to treatment program, if necessary. Subjects were then given a 10-14 day rest period after which a challenge or retest dose was applied once to a previously unexposed test site. The retest dose was equivalent to any one of the original nine applications. Reactions were scored 24 and 48 hours after application. Comparison was made between the nine inductive responses and the retest dose.

No adverse reactions of any kind were observed during the course of the study. It was concluded that the reversible oral adhesive gel is a “non-primary irritant” and a “non-primary sensitizer”. Individual subject results are detailed in Table 4.

TABLE 4
SUMMARY OF RESULTS
SEMI-OCCLUSIVE PATCH
CR Lab No.: H0219-I
Client No.: LipZip

| | Subject | | | Response | | | | | | | | | Chall. | |
|---|---|---|---|---|---|---|---|---|---|---|---|---|---|---|
| No. | ID | RACE | SEX | 1 | 2 | 3 | 4 | 5 | 6 | 7 | 8 | 9 | 24 HR | 48 HR |
| 1 | 03-7237 | C | F | 0 | 0 | 0 | 0 | 0 | 0 | 0 | 0 | 0 | 0 | 0 |
| 2 | 03-7246 | H | F | 0 | 0 | 0 | 0 | 0 | 0 | 0 | 0 | 0 | 0 | 0 |
| 3 | 03-7238 | C | F | 0 | 0 | 0 | 0 | 0 | 0 | 0 | 0 | 0 | 0 | 0 |
| 4 | 03-6500 | AA | F | 0 | 0 | 0 | 0 | 0 | 0 | 0 | 0 | 0 | 0 | 0 |
| 5 | 03-7546 | AA | F | 0 | 0 | 0 | 0 | 0 | 0 | 0 | 0 | 0 | 0 | 0 |
| 6 | 03-8068 | AA | F | 0 | 0 | 0 | 0 | 0 | 0 | 0 | 0 | 0 | 0 | 0 |
| 7 | 03-8112 | AA | F | 0 | 0 | 0 | 0 | 0 | 0 | 0 | 0 | 0 | 0 | 0 |
| 8 | 03-8113 | AA | M | 0 | 0 | 0 | 0 | 0 | 0 | 0 | 0 | 0 | 0 | 0 |
| 9 | 03-6668 | C | F | 0 | 0 | 0 | 0 | 0 | 0 | 0 | 0 | 0 | 0 | 0 |
| 10 | 03-6076 | C | F | 0 | 0 | 0 | 0 | 0 | 0 | 0 | 0 | 0 | 0 | 0 |
| 11 | 03-6256 | C | F | 0 | 0 | 0 | 0 | 0 | 0 | 0 | 0 | 0 | 0 | 0 |
| 12 | 03-6805 | A | M | 0 | 0 | 0 | 0 | 0 | 0 | 0 | 0 | 0 | 0 | 0 |
| 13 | 03-7657 | AA | M | 0 | 0 | 0 | 0 | 0 | 0 | 0 | 0 | 0 | 0 | 0 |
| 14 | 03-7576 | C | F | 0 | 0 | 0 | 0 | 0 | 0 | 0 | 0 | 0 | 0 | 0 |
| 15 | 03-6045 | A | M | 0 | 0 | 0 | 0 | 0 | 0 | 0 | 0 | 0 | 0 | 0 |
| 16 | 03-7504 | AA | F | 0 | 0 | 0 | 0 | 0 | 0 | 0 | 0 | 0 | 0 | 0 |
| 17 | 03-7617 | C | F | 0 | 0 | 0 | 0 | 0 | 0 | 0 | 0 | 0 | 0 | 0 |
| 18 | 03-7269 | C | F | 0 | 0 | 0 | 0 | 0 | 0 | 0 | 0 | 0 | 0 | 0 |
| 19 | 03-6176 | AA | F | 0 | 0 | 0 | 0 | 0 | 0 | 0 | 0 | 0 | 0 | 0 |
| 20 | 03-6933 | AA | M | 0 | 0 | 0 | 0 | 0 | 0 | 0 | 0 | 0 | 0 | 0 |
| 21 | 03-6573 | AA | M | 0 | 0 | 0 | 0 | 0 | 0 | 0 | 0 | 0 | 0 | 0 |
| 22 | 03-7029 | AA | F | 0 | 0 | 0 | 0 | 0 | 0 | 0 | 0 | 0 | 0 | 0 |
| 23 | 03-6996 | AA | F | 0 | 0 | 0 | 0 | 0 | 0 | 0 | 0 | 0 | 0 | 0 |
| 24 | 03-7693 | AA | M | 0 | 0 | 0 | 0 | 0 | 0 | 0 | 0 | 0 | 0 | 0 |
| 25 | 03-7469 | C | M | 0 | 0 | 0 | 0 | 0 | 0 | 0 | 0 | 0 | 0 | 0 |
| 26 | 03-6100 | C | F | 0 | 0 | 0 | 0 | 0 | 0 | 0 | 0 | 0 | 0 | 0 |
| 27 | 03-7050 | AA | F | 0 | 0 | 0 | 0 | 0 | 0 | 0 | 0 | 0 | 0 | 0 |
| 28 | 03-7634 | AA | M | 0 | 0 | 0 | 0 | 0 | 0 | 0 | 0 | 0 | 0 | 0 |
| 29 | 03-6163 | AA | F | 0 | 0 | 0 | 0 | 0 | 0 | 0 | 0 | 0 | 0 | 0 |
| 30 | 03-6565 | C | F | 0 | 0 | 0 | 0 | 0 | 0 | 0 | 0 | 0 | 0 | 0 |
| 31 | 03-6509 | AA | F | 0 | 0 | 0 | 0 | 0 | 0 | 0 | 0 | 0 | 0 | 0 |
| 32 | 03-7680 | AA | F | 0 | 0 | 0 | 0 | 0 | 0 | 0 | 0 | 0 | 0 | 0 |
| 33 | 03-8067 | AA | F | 0 | 0 | 0 | 0 | 0 | 0 | 0 | 0 | 0 | 0 | 0 |
| 34 | 03-8080 | AA | F | 0 | 0 | 0 | 0 | 0 | 0 | 0 | 0 | 0 | 0 | 0 |
| 35 | 03-7744 | H | F | 0 | 0 | 0 | 0 | 0 | 0 | 0 | 0 | 0 | 0 | 0 |
| 36 | 03-8066 | AA | F | 0 | 0 | 0 | Dc | Dc | Dc | Dc | Dc | Dc | Dc | Dc |
| 37 | 03-7079 | C | F | 0 | 0 | 0 | 0 | 0 | 0 | 0 | 0 | 0 | 0 | 0 |
| 38 | 03-6034 | C | F | 0 | 0 | 0 | 0 | 0 | 0 | 0 | 0 | 0 | 0 | 0 |
| 39 | 03-6770 | C | F | 0 | 0 | 0 | 0 | 0 | 0 | 0 | 0 | 0 | 0 | 0 |
| 40 | 03-6257 | C | F | 0 | 0 | 0 | 0 | 0 | 0 | 0 | 0 | 0 | 0 | 0 |
| 41 | 03-6929 | C | F | 0 | 0 | 0 | 0 | 0 | 0 | 0 | 0 | 0 | 0 | 0 |
| 42 | 03-7080 | C | F | 0 | 0 | 0 | 0 | 0 | 0 | 0 | 0 | 0 | 0 | 0 |
| 43 | 03-7597 | AA | F | 0 | 0 | 0 | 0 | 0 | 0 | 0 | 0 | 0 | 0 | 0 |
| 44 | 03-6718 | C | F | 0 | 0 | 0 | 0 | 0 | 0 | 0 | 0 | 0 | 0 | 0 |
| 45 | 03-7002 | C | F | 0 | 0 | 0 | 0 | 0 | 0 | 0 | 0 | 0 | 0 | 0 |
| 46 | 03-6643 | C | F | 0 | 0 | 0 | 0 | 0 | 0 | 0 | 0 | 0 | 0 | 0 |
| 47 | 03-7261 | H | F | 0 | 0 | 0 | 0 | 0 | 0 | 0 | 0 | 0 | 0 | 0 |
| 48 | 03-7251 | C | F | 0 | 0 | 0 | 0 | 0 | 0 | 0 | 0 | 0 | 0 | 0 |
| 49 | 03-7250 | C | F | 0 | 0 | 0 | 0 | 0 | 0 | 0 | 0 | 0 | 0 | 0 |
| 50 | 03-7493 | C | M | 0 | 0 | 0 | 0 | 0 | 0 | 0 | 0 | 0 | 0 | 0 |
| 51 | 03-8041 | H | F | 0 | 0 | 0 | 0 | 0 | 0 | 0 | 0 | 0 | 0 | 0 |
| 52 | 03-7551 | AA | M | 0 | 0 | 0 | 0 | 0 | 0 | 0 | 0 | 0 | 0 | 0 |
| 53 | 03-7585 | AA | M | 0 | 0 | 0 | 0 | 0 | 0 | 0 | 0 | 0 | 0 | 0 |
| 54 | 03-7366 | AA | F | 0 | 0 | 0 | 0 | 0 | 0 | 0 | 0 | 0 | 0 | 0 |
| 55 | 03-8096 | AA | F | 0 | 0 | 0 | 0 | 0 | 0 | 0 | 0 | 0 | 0 | 0 |
| 56 | 03-6970 | C | M | 0 | 0 | 0 | 0 | 0 | 0 | 0 | 0 | 0 | 0 | 0 |
| 57 | 03-6919 | AA | F | 0 | 0 | 0 | 0 | 0 | 0 | 0 | 0 | 0 | 0 | 0 |
| 58 | 03-7461 | AA | F | 0 | 0 | 0 | 0 | 0 | 0 | 0 | 0 | 0 | 0 | 0 |

Definition of Symbols Shown in Table 4:
0—No evidence of any effect;
?—(Barely perceptible) minimal faint (light pink) uniform or spotty erythema;
1—(Mild) pink uniform erythema covering most of contact site;
2—(Moderate) pink\red erythema visibly uniform in entire contact area;
3—(Marked) bright red erythema with accompanying edema, petechiae or papules;
4—(Severe) deep red erythema with vesiculation or weeping with or without edema;
D—Patch eliminated due to reaction;
Dc—Discontinued due to absence of subject on application date;
M—Patch applied to an adjacent site after strong test reaction;
S—Skin stained from pigment in product;
T—Tan
Note:
Results were recorded by technicians who had taken and passed a modified visual discrimination examination conducted by a Board Certified Ophthalmologist (Farnsworth-Munsell 100 Hue Test, which determines a person's ability to discern color against a black background, modified to include a flesh tone background more nearly approaching actual use conditions, wherein erythematous skin is graded according to intensity).

### Example 4

### Cytotoxicity Studies of the First Preferred Embodiment

Cytotoxicity studies were performed by ams Laboratories Pty Ltd of Silverwater, NSW, Australia according to ISO 10993-5 (2002) and AS/NZS 26961996. The reversible oral adhesive gel was found to be non-cytotoxic.

The following protocol was used to test cytotoxicity:

### Materials and Methods

### Cell Lines and Media

The cells used in the study were Vero, obtained from the ATCC. Vero is a standard cell line for use in cytotoxicity testing. The cells were grown and maintained in Eagle's minimal essential medium (EMEM) containing L-glutamine and Hepes buffer, 10% by volume PBS. Agar medium was prepared with one part of double concentration of sterile complete culture medium plus one part of sterile 2% agarose in water for irrigation. Melted agar and medium were brought to 42° C. in a water bath and mix aseptically.

### Cytotoxicity Assay

A working stock of Vero cells in suspension was used to seed the 6-well plates used, which were then incubated in a humidified 5% CO2 incubator at 37° C. until the cells were confluent. Once confluent the medium was aspirated and 2.5 ml of agar medium was added to each well. The agarose was allowed to solidify for approximately 10 minutes at room temperature followed by 30 min in the incubator.

50 μl of sample was diluted in 200RI EMEM (2×) (this was considered to be 100% concentrate). A further 66% concentration was made and also used for the test. 50 μl of the prepared sample (in triplicate) was dispensed by spreading onto the solidified agarose surface and incubated for 48 hours at 37° C. in a humidified 5% CO₂ incubator.

Known cytotoxic and non-cytotoxic materials (in triplicate) were used as positive and negative controls. Three wells with EMEM (2×), medium plus another three wells with overlay agar were included as media controls.

After 48 hours of incubation, sample was aspirated and 2.5 ml of neutral red stain solution in sterile PBS was dispensed onto the solidified agarose surface and incubated for 30 min at 37° C. in the dark before aspirating the excess stain solution. Cells were then examined using an inverted microscope.

Results of the cytotoxicity testing are shown in Table 5.

TABLE 5
Cytotoxicity Testing of Oral Adhesive Gel and controls

| | Cytotoxicity Scale | |
|---|---|---|
| Treatment group | (Individual Results) | Interpretation |
| Oral Adhesive Gel | 0, 0, 0 | Non Cytotoxic |
| (100%) | | |
| Oral Adhesive Gel | 0, 0, 0 | Non cytotoxic |
| (66%) | | |
| Positive controls | 3, 3, 3 | Severely cytotoxic |
| Negative controls | 0, 0, 0 | Non cytotoxic |
| EMEM medium control | 0, 0, 0 | Non cytotoxic |

The scoring system of Table 5 is described in Table 6.

TABLE 6
Evaluation Criteria

| Cytotoxicity Scale | Interpretation | Cell lysis |
|---|---|---|
| 0 | Non-cytotoxic | Not more than 20% |
| 1 | Mildly cytotoxic | Not more than 50% |
| 2 | Moderately cytotoxic | Not more than 70% |
| 3 | Severely cytotoxic | More than 75% |
| 4 | Complete cytotoxic | More than 90% |

According to the results in Table 5, the oral adhesive gel proved to be non-cytotoxic by the indirect contact method based on AS ISO 10993.5-2002 and AS/NZS 2696:1996.

### Example 5

### Second Preferred Embodiment (Multiple Adhesives)

A preferred embodiment of the present invention incorporating more than one adhesive agent was prepared with the ingredients shown in Table 7:

TABLE 7

| w/w % | gm | material | Function | supplier |
|---|---|---|---|---|
| 54.7 ie q.s to | 547 | Purified water | Diluent | |
| 100 w/w % | | | | |
| 0.1 | 1 | Disodium | Chelating agent | IMCD |
| | | Edetate | | |
| 0.2 | 2 | Potassium | Preservative | IMCD |
| | | Sorbate | | |
| 2.0 ie q.s. to | 20 | Citric Acid | pH adjuster | APS |
| pH <5.0 | | Anhydrous | | Chemicals |
| 12.0 | 120 | Gantrez ® | Adhesive | ISP |
| | | MS-955 | | |
| 10.0 | 100 | Ethanol 95% | Solvent | CSR |
| | | Undenatured | | Distilleries |
| 12.0 | 120 | Kollidon 90F | Adhesive | Ingredients |
| | | | | Plus |
| 2.0 | 20 | Xanthan Gum | Thickener/gelling | Bronson & |
| | | | agent | Jacobs |
| 4.5 | 45 | Dimethicone | Lubricant | Ingredients |
| | | 200 | | Plus |
| 1.0 | 10 | Aerosil ® 200 | Thickener | Chemiplas |
| 1.5 | 15 | Sepigel ® 305 | Thickener | Bronson & |
| | | | | Jacobs |

With reference to the above table, Gantrez ® MS-955 is a calcium/sodium PVM/MA copolymer, Kollidon ® 90F is a soluble polyvinyl pyrrolidone, Dimethicone 200 is DC silicone fluid 200/200, Aerosil ® 200 is silicon dioxide and Sepigel ® 305 is the combination of polyacrylamide, C₁₃-C₁₄ isoparaffin and laureth-7. Ethanol 95% must be undenatured.

Disodium edetate, potassium sorbate and citric acid were dissolved in the purified water. The Gantrez® MS-955 was added and mixed until totally dispersed avoiding any lump formation. At this stage the batch began to thicken. The undenatured ethanol was then added with mixing until uniformly dispersed. The Kollidon 90F was then added with mixing until uniformly dispersed with care taken to prevent lump formation. Xanthan gum was then added and mixed until uniform avoiding any lump formation. It was noted that the batch was further thickened by this addition. Dimethicone was then added until uniform, followed by the addition of Aerosil® 200 (with further thickening of the batch) and finally by the addition of Sepigel® 305 which was mixed until uniform (again with further thickening of the batch).

A detailed description of a preferred embodiment of the reversible oral adhesive gel incorporating multiple adhesives according to the invention is as follows:

Components
Purified Water (carrier): 54.7%
Disodium Edetate (chelating agent): 0.1%
Potassium Sorbate (preservative): 0.2%
Citric Acid Anhydrous (pH adjuster): 2%
Gantrez MS-955 [calcium/sodium PVM/MA copolymer] (adhesive): 12%
Ethanol 95% undenatured (solvent): 10%
Kollidon 90F [soluble polyvinyl pyrrolidone] (adhesive): 12%
Xanthum Gum (thickener): 2%
Dimethicone 200 [DC Silicone Fluid 200/200] (lubricant): 4.5%
Aerosil 200 [Silicon Dioxide] (thickener): 1%
Sepigel 305 [Polyacrylamide and C13-14 Isoparaffin and Laureth-7] (thickener): 1.5%

Method
1. Dissolve the Disodium Acetate, Potassium Sorbate and Citric Acid in the Purified Water.
2. Slowly sprinkle the Gantrez MS-955 into the batch and mix until totally dispersed. Avoid lump formation. Batch will begin to thicken.
3. Add the Ethanol with mixing until uniform. NB: Ethanol must be undenatured.
4. Disperse the Kollidon 90F with mixing until uniform. Avoid lump formation.
5. Disperse the Xanthum Gum with mixing until uniform. Avoid lump formation. Batch will thicken further.
6. Add the Dimethicone 200 and mix until uniform.
7. Add the Aerosil 200 and mix until uniform. Batch will thicken further.
8. Add the Sepigel 305 and mix until uniform. Batch thickens even further.
Final pH should be 5.0 or below.

The resulting product was a thick, pale straw coloured, slightly translucent gel with a slightly gum like, otherwise bland odour. The pH of the gel was 4.0 to 5.0 (1 in 5 dilution with water) and the viscosity was about 1 million cps (as determined using a Brookfield RVT Helipath Spindle F, 5 rpm).

The reversible oral adhesive gels of Examples 1 and 5 both provided the desired adhesion to the lips. The reversible oral adhesive gel of Example 5 was thicker than that of Example 1, indicating a synergistic increase in viscosity with the addition of the second adhesive. Although Xanthan gum was employed for thickening in both the single adhesive formulation (Example 1: Gantrez® MS-955 alone) and the multiple adhesive formulation (Example 5: Gantrez® MS-955+Kollidon 90F), it was surprisingly and unexpectedly found that there was thickening synergy between the two adhesives, that enabled the quantity of Xanthan gum to be reduced in Example 5 while simultaneously achieving increased viscosity and adhesion. The addition of ethanol in Example 5 allowed the drying time to be speeded up compared to the drying time in Example 1, wherein ethanol was not used.

While the disclosure has been illustrated and described in detail in the foregoing description and examples, this disclosure should be considered to be illustrative only and not restrictive, it being understood that only preferred embodiments are described and all modifications that come within the spirit of the disclosure are desired to be protected.

1. A reversible oral adhesive gel, the oral adhesive comprising one or more adhesive agents, the oral adhesive in the form of a gel and having a pH of 5.0 or lower.

2. The reversible oral adhesive gel according to claim 1, wherein the oral adhesive has an average maximum stress value ranging from about 0.03 to about 0.1 MPa.

3. The reversible oral adhesive gel according to claim 1, wherein the adhesive agent or agents are selected from the group consisting of: an alkyl vinyl ether/maleic anhydride copolymer, mixed sodium/calcium salts of methyl vinyl ether/maleic anhydride copolymer and soluble polyvinyl pyrrolidones.

4. The reversible oral adhesive gel according to claim 1, wherein the adhesive agent or agents are present in an amount of 5 to 25 wt %.

5. The reversible oral adhesive gel according to claim 1, further comprising one or more of:
one or more chelating agents;
one or more preservatives;
one or more pH adjusters;
one or more hydrophilic gelling/thickening agents;
one or more additional thickeners;
one or more lubricants;
one or more solvents; and/or
one or more diluents.

6. The reversible oral adhesive gel according to claim 1, further comprising about 0.01 to 0.2 wt % of chelating agent(s), about 0.01 to 1 wt % of preservative(s), sufficient amount of a pH adjuster to obtain a final pH of the composition of 5.0 or lower, about 1 wt % to 10 wt % of thickener/hydrophilic gelling agent(s), about 1 to 10 wt % of additional thickener(s), about 1 to about 10 wt % of lubricant(s), about 2 to about 20% solvents, with the balance being one or more diluents.

7. The reversible oral adhesive gel according to claim 1, wherein the adhesive agent is mixed sodium/calcium salts of methyl vinyl ether/maleic anhydride copolymer and the adhesive further comprises xanthan gum as a hydrophilic gelling agent.

8. The reversible oral adhesive gel according to claim 1, wherein said reversible oral adhesive gel is contained within a tube composed of a body or reservoir for storing said gel and an extended nozzle to facilitate application of said gel onto lips.

9. A method of treating a patient, the method comprising applying a reversible oral adhesive to the lips to secure the patient's lips together to inhibit the patient breathing through the mouth and to encourage the patient to breathe through the nose, wherein the oral adhesive comprises one or more adhesive agents, and wherein the oral adhesive is in the form of a gel and has a pH of 5.0 or lower.

10. The method according to claim 9, wherein the oral adhesive has an average maximum stress value ranging from about 0.03 to about 0.1 MPa.

11. The method according to claim 9, wherein the adhesive agent or agents are selected from the group consisting of: an alkyl vinyl ether/maleic anhydride copolymer, mixed sodium/calcium salts of methyl vinyl ether/maleic anhydride copolymer and soluble polyvinyl pyrrolidones.

12. The method according to claim 9, wherein the adhesive agent or agents are present in an amount of 5 to 25 wt %.

13. The method according to claim 9, further comprising one or more of:
one or more chelating agents;
one or more preservatives;
one or more pH adjusters;
one or more hydrophilic gelling/thickening agents;
one or more additional thickeners;
one or more lubricants;
one or more solvents; and/or
one or more diluents.

14. The method according to claim 9, further comprising about 0.01 to 0.2 wt % of chelating agent(s), about 0.01 to 1 wt % of preservative(s), sufficient amount of a pH adjuster to obtain a final pH of the composition of 5.0 or lower, about 1 wt % to 10 wt % of thickener/hydrophilic gelling agent(s), about 1 to 10 wt % of additional thickener(s), about 1 to about 10 wt % of lubricant(s), about 2 to about 20% solvents, with the balance being one or more diluents.

15. The method according to claim 9, wherein the adhesive agent is mixed sodium/calcium salts of methyl vinyl ether/maleic anhydride copolymer and the adhesive further comprises xanthan gum as a hydrophilic gelling agent.

16. The method according to claim 9, wherein said reversible oral adhesive gel is contained within a tube composed of a body or reservoir for storing said gel and an extended nozzle to facilitate application of said gel onto lips.

17. The method according to claim 9, wherein said lips are secured by said adhesive, and further comprising contacting the secured lips from within with saliva so as to release the lips from each other.

18. The method of claim 17, wherein saliva contacts the secured lips from within so as to release of said lips from each other, and further comprising resealing of said lips without the need for additional oral adhesive gel.

19. A kit comprising the oral adhesive gel of claim 1 and packaging materials therefore.

20. The kit of claim 19, wherein the oral adhesive is contained in a tube.

21. The kit of claim 20, wherein the tube comprises aluminum or aluminum barrier laminate.

22. The kit of claim 19, wherein the tube comprises a plastic screw cap.

23. The kit of claim 19, wherein the tube comprises a nozzle.

24. The kit of claim 23, wherein the nozzle is 6 mm to 12 mm in length.

25. A method of preparing a reversible oral adhesive gel, comprising the steps of:
a) mixing 1.0 gm of Disodium Edetate, 2.0 gm of Potassium Sorbate and 20.0 gm of Citric Acid Anhydrous in 547.0 gm of Purified Water for 10 min at 25° C.;
b) adding to the mixture of step (a) 120.0 gm of Gantrez MS-955, avoiding lump formation and mixing until fully dispersed;
c) adding to the mixture of step (b) 100.0 gm of Ethanol 95% Undenatured mixing until fully dispersed;
d) adding to the mixture of step (c) 120.0 gm of Kollidon 90F avoiding lump formation and mixing until fully dispersed;
e) adding to the mixture of step (d) 20.0 gm of Xanthum Gum, avoiding lump formation and mixing until fully dispersed;
f) adding to the mixture of step (e) 45.0 gm of Dimethicone 200, avoiding lump formation and mixing until fully dispersed;
g) adding to the mixture of step (f) 10.0 gm of Aerosil 200, avoiding lump formation and mixing until fully dispersed;
h) adding to the mixture of step (g) 15.0 gm of Sepigel 305, avoiding lump formation and mixing until fully dispersed;
thereby forming said reversible oral adhesive gel.
